(19) United States
(12) Patent Application Publication
(10) Pub. No.: US 2013/0344180 A1
(43) Pub. Date: Dec. 26, 2013

(54) WEIGHT LOSS AND SATIATION COMPOSITION

(71) Applicant: Marc Schreuder, Provo, UT (US)

(72) Inventor: Marc Schreuder, Provo, UT (US)

(73) Assignee: YL HOLDINGS, INC., Lehi, UT (US)

(21) Appl. No.: 13/533,684

(22) Filed: Jun. 26, 2012

(51) Int. Cl. A61K 36/482; A61P 3/00; A61K 31/20; A61K 36/899; A61K 36/54; A61K 36/00

(52) U.S. Cl. 424/739; 424/725; 424/773; 514/560

(57) ABSTRACT

Weight loss compositions and systems are disclosed that may include various combinations of essential oils and other components, which may be effective in promoting weight loss. Some implementations may include a fat blocker, a filler component, an insulin sensitizer, and a fat growth suppressant. In some instances, the fat blocker may include a carotenoid. In other instances, the fat blocker may be selected from the group consisting of fucoxanthin and punicic acid. A weight loss composition may include a first component and a second component, wherein the combination of the first and second components may be effective in promoting weight loss. The first element may include a fat blocker, a filler component, and a first insulin sensitizer. The second element may comprise a fat growth suppressant and a second insulin sensitizer.

### FIELD

The present disclosure relates to a weight loss composition. Specifically, this disclosure relates to a chemical composition comprising various combinations of essential oils and other compounds, the combination of which may be effective in promoting weight loss and feelings of satiation. The present disclosure further relates to a weight loss and satiation composition comprising a first component and a second component, wherein the combination of the first and second components may be effective in promoting weight loss.

### BACKGROUND

In recent years, sales for essential oils and other topically applied oils have rapidly increased. Topically applied oils are usually oils which are derived from, or include certain natural essential components or essences of different substances from plants. Such topically applied oils are generally referred to as essential oils.

Essential oils are commonly known asnature's living energy, and as such are the natural aromatic volatile liquids found in shrubs, flowers, trees, resins, fruit peels, rhizomes, roots, bushes, and seeds. The distinctive components in essential oils defend plants against insects, environmental conditions, and disease. They are also vital for a plant to grow, live, evolve, and adapt to its surroun Essential Hs are extracted from aromatic plant sources via steam distillation, cold pressing, and other types of distillation. Essential oils are highly concentrated and far more potent than dry herbs. Other topically applied oils and fatty oils may include olive oil, almond oil, coconut oil, etc., and oils high in fats and esters, such as jojoba oil, and waxes such as beeswax.

While essential oils often have a pleasant aroma, their phytochemical makeup is complex and their benefits vast. Historically, essential oils have played a prominent role in everyday life. With more than 200 references to aromatics, incense, and ointments throughout the Bible, essential oils are said to be used for anointing and healing the sick. Today, essential oils are commonly used for aromatherapy, massage therapy, emotional health, personal care, nutritional supplements, household solutions, and much more.

For example, essential oils have been used as active ingredients in various types of prescription, over-the-counter, and consumer products which are designed to provide various benefits. Essential oils have also been used as agents to improve the mental state of a user, thereby placing the user in a mental condition that is conducive to self-control and appetite control.

While brain chemistry and mental conditioning are known to play a role in weight loss, there are many other physical, chemical and emotional factors that contribute to weight loss. For example, a large body of literature shows that many phytochemicals and natural products are used to treat obesity as metabolic stimulants, appetite suppressants, and starch blockers and by regulation of glucose and lipid metabolism, as well as targeting adipocyte differentiation. Currently available weight loss compositions address some of these factors, but fail to address others. Thus, while compositions currently exist which are designed to promote weight loss, challenges still exist. Accordingly, it would be an improvement in the art to augment or even replace current techniques with other techniques and/or compositions.

### BRIEF SUMMARY

The present disclosure relates to a weight loss and satiation composition. Specifically, this disclosure relates to a chemical composition that may include various combinations of essential oils, which combinations may be effective in promoting weight loss. The present disclosure further relates to a weight loss and satiation composition that may include a first component and a second component, wherein the combination of the first and second components may be effective in promoting weight loss.

Some implementations may provide a composition for promoting weight loss, which includes a fat blocker, a filler component, an insulin sensitizer, and a fat growth suppressant. In some instances, the fat blocker may include a carotenoid. In other instances, the fat blocker may be selected from the group consisting of fucoxanthin and punicic acid.

Some implementations may further provide a filler component which includes a high-fiber composition. An example of a high-fiber composition may include konjac root and/or glucomannan. An insulin sensitizer component may be further provided and selected from a group consisting of cinnamon, Cinnamonum cassia, cassia, ocotea, and punicic acid. In some instances an insulin sensitizer may be further selected from a group consisting of ocotea, cloves, bay leaves, turmeric, bitter melon, Gymnema, Korean ginsing, onions, garlic, flaxseed meal, α-lipoic acid, biotin, L-carnitine, vanadium, chromium, magnesium, zinc, vitamin B₃, vitamin E, and vitamin K. Some aspects may further include a fat growth suppressant the selected from the group consisting of citral and lemongrass.

Some implementations may further provide a weight loss system which includes a first element and a second element. The first and second elements may be selected to optimize appetite suppression and caloric metabolism for user. In some instances, a first element may be provided which includes a fat blocker, a filler component, and a first insulin sensitizer. A second element may be further provided which includes a fat growth suppressant, and a second insulin sensitizer. In some embodiments, the first and second insulin sensitizers may include punicic acid. In other embodiments, the first and second insulin sensitizers may include one or more of cloves, bay leaves, turmeric, frankincense resin, boswellic acids, bitter melon, Gymnema, Korean ginsing, onions, garlic, coconut oil (or other medium chain transglycerides), flaxseed meal, α-lipoic acid, biotin, L-carnitine, vanadium, chromium, magnesium, zinc, vitamin B₃, vitamin E, and vitamin K, or other known insulin sensitizers. The various ingredients may in any useful form, such as powdered, distilled as an oil, chopped leaf, etc.

Some implementations of the present invention may further include a weight loss kit which includes a first element and a second element, wherein the weight loss kit further includes a diet plan whereby a user administers the first element prior to ingesting a meal, and whereby the user administers the second element during or after the consumption of the meal. The weight loss kit may further include instructions and/or guidelines for optimizing use of the weight loss kit.

### DETAILED DESCRIPTION

The present disclosure relates to a weight loss and satiation composition and/or kit. Throughout this specification, there are ranges defined by upper and lower boundaries. Each lower boundary can be combined with each upper boundary to define a range. The lower and upper boundaries should each be taken as a separate element.

In general, embodiments of weight loss and satiation compositions and/or kits may provide certain formulae and combinations of formulae which promote weight loss. Some aspects may provide various nutraceutical compositions which may include various essential oils, plants, herbs, extracts, spices, and pigments which have been found to be effective in promoting weight loss. Thus, while specific examples of known nutraceutical compositions and combined nutraceutical therapies are discussed, one having skill in the art will appreciate that the specific components may be substituted with natural or synthetic equivalents to achieve a desired results within the spirit and teaching of weight loss and satiation compositions and/or kits. Accordingly, the specific embodiments provided herein are not intended to be limiting, but rather are provided as a means for disclosing and teaching underlying principles.

Some embodiments may provide a weight loss and satiation composition comprising 1) a fat blocker, 2) a filler component, 3) an insulin sensitizer, and 4) a fat growth suppressant. Some embodiments may further include a cellular energy enhancer or metabolic acceleration compound. Similarly, some embodiments may further provide a nutraceutical product that is effective in promoting weight loss. The nutraceutical product may include various combinations of active and inactive ingredients, wherein the final product promotes weight loss. In some instances, a nutraceutical product may be provided comprising a two-part system, wherein the benefit of weight loss is realized by taking both parts of the two-part system. The various components of the nutraceutical or weight loss products are discussed below.

### Weight Loss Composition Component Overview

Fat Blocker Component

Some exemplary weight loss compositions may include at least one fat blocker component. A fat block component may be understood to include any naturally occurring or naturally derived component or composition that demonstrates anti-obesity potential, evidenced in the suppression of adipocyte differentiation and high-fat-diet induced obesity, and/or insulin resistance in vivo. For example, in one embodiment a weight loss composition may be provided which includes a carotenoid, such as fucoxanthin.

Carotenoids are tetraterpenoid organic pigments that are naturally occurring in the chloroplast and chromoplasts of plants and some other photosynthetic organisms, such as algae, some types of bacteria, and some types of fungus. Carotenoids may further include synthesized fats and other basic organic metabolic building blocks by all these organisms. There are over 600 known carotenoids. Accordingly, one having skill in the art will appreciate that the specific examples provided herein may be substituted with various known carotenoids to achieve the novel benefits taught herein.

In some embodiments, a weight loss composition may be provided which comprises fucoxanthin as a fat blocker component. Fucoxanthin is a xanthophyll, with formula C₄₂H₅₈O₆. Fucoxanthin is found as an accessory pigment in the chloroplasts of brown algae and most other heterokonts, giving them a brown or olive-green color. Fucoxanthin absorbs light primarily in the blue-green to yellow-green part of the visible spectrum, peaking at around 510-525 nm by various estimates and absorbing significantly in the range of 450 to 540 nm. Fucoxanthin has been shown to promote fat burning within fat cells in white adipose tissue by increasing the expression of thermogenin.

A fat blocker component in accordance with the present invention may further include punicic acid. Punicic acid may be a conjugated linolenic acid, rich in the seeds of Punica granatum (about 70%), which is reported to inhibit cancer cell proliferation, neutrophil hyper- activation, and colonic inflammation in vivo. Recent research has also demonstrated the anti-obesity potential of punicic acid, evidenced in the suppression of adipocyte differentiation and high-fat-diet induced obesity and insulin resistance in vivo. Accordingly, some embodiments may comprise a weight loss composition which includes punicic acid as a fat blocker component.

Filler Component

Some exemplary weight loss and satiation compositions may comprise at least one filler component. A filler component may be understood to include any naturally occurring or naturally derived component or composition that expands or increases in bulk within a user's stomach when ingested, thereby trigerring feelings of satiation. Thus, in some embodiments a filler component acts as an appetite suppressant to curb the user's desires for food.

In some embodiments, a filler component may include a high-fiber composition capable of absorbing a great amount of water. For example, some embodiments may include a filler component with at least some konjac root. Konjac root comes from the Asian plant Amorphophallus konjac and is known as the source of polysaccharide glucomannan. Accordingly, some embodiments may further comprise a filler component selected from glucomannan.

Konjac root is commonly used to promote weight loss, but also as a form of blood sugar control. Since it is so high in fiber, it may even be able to lower cholesterol levels and reduce the absorption of fat. Konjac root can absorb a great amount of water, so it increases in volume when invoked inside the stomach, thereby working to make the user feel full. In some embodiments, hydroxypropyl methylcellulose may be included to improve the effectiveness of the filler. Additionally, the filler may function as a dispersant to evenly distribute the other components when consumed.

Insulin Sensitizer Component

Some exemplary weight loss compositions may further include at least one insulin sensitizer. An insulin sensitizer may be understood to include any naturally occurring or naturally derived component or composition that demonstrates a reduction of serum glucose in a user. An insulin sensitizer may further be understood to include any naturally occurring or naturally derived component or composition that demonstrates a reduction in triglycerides, LDL cholesterol, and total cholesterol serum levels in a user. An insulin sensitizer in accordance with the present invention may further include any naturally occurring or naturally derived component or composition which promotes increased sensitivity to insulin, and results in improved energy metabolism.

Some embodiments may include an insulin sensitizer comprising cinnamon. Cinnamon (Cinnamonum cassia) has been shown to reduce serum glucose, triglycerides, LDL cholesterol, and total cholesterol in people with Type II diabetes. Cinnamon may be delivered in any suitable form, such as an essential oil, extract, bark, leaf, etc. Additional spices including cloves, bay leaves, and turmeric further display insulin enhancing activity in vitro. Botanical products have also been shown to improve glucose metabolism by hypoglycemic effect and also by improving lipid metabolism, antioxidant status, and capillary function. A number of medicinal/culinary herbs have also been reported to yield hypoglycemic effect in patients with diabetes. Examples of these include bitter melon, Gymnema, Korean ginseng, onions, garlic, ocotea, flaxseed meal, and specific nutrients including α-lipoic acid, biotin, L-carnitine, vanadium, chromium, magnesium, zinc, and vitamins B₃, E, and K. Accordingly, various embodiments may include any component or combination of components which have been shown to act as an insulin sensitizer, including essential oils of those components.

Some embodiments may further include an insulin sensitizer comprising punicic acid. Some studies suggest that punicic acid provides an insulin sensitizing effect, wherein punicic acid acts as a ligand of PPARγ, which plays a crucial role in adipocyte differentiation. Accordingly, some weight loss compositions of the present invention utilize punicic acid as both an insulin sensitizer component and a fat blocker component.

Fat Growth Suppressant Component

Some embodiments of weight loss compositions may further include at least one fat growth suppressant component. A fat growth suppressant component may be understood to include any naturally occurring or naturally derived component or composition that suppresses differentiation of fibroblasts into adipocytes and slows down maturation.

In some embodiments, a fat growth suppressant component may include citral. Citral is a naturally occurring aliphatic aldehyde of the terpene series, and comprises a mixture of cis and trans isomers (geranial and neral, respectively) of 3,7-dimethyl-2,6-octadienal. Geranial and neral are the main components (approximately 80%) of lemongrass oil, and are found in all citrus fruits and used extensively in the food, cosmetic, and detergent industries. Lemongrass (Cymbopogon citrates, Cymbopogon flexuosus) is a prime commercial source of citral. Lemongrass is an evergreen plant that grows widely in Asia and is used in Asian households as an herb. Lemongrass is known to possess antiseptic, antimicrobial, anti-inflammatory, carminative, diuretic, and central nervous system-stimulating effects. Citral has also been shown to be effective against prostate gland tumors in various strains of rats. Citral is non-toxic and does not possess carcinogenic potential in mice and rats. Similarly, Lemon Myrtle oil may also provide a high-level source of citral.

Citral competitively inhibits E1, E2, and E3 isozymes of retinaldehyde dehydrogenase (Raldh), thereby increasing retinaldehyde concentrations in the adipose tissue. Reginaldehyde suppresses differentiation of fibroblasts into adipocytes and slows down maturation. Accordingly, citral has been shown effective as a fat growth suppressant component. Similarly, lemongrass oil has been shown effective as a fat growth suppressant component. One having skill in the art will appreciate that any naturally occurring or naturally derived composition or material high in citral may be substituted as a fat growth suppressant component. For example, citral is present in the oils of several plants, including lemon myrtle (Backhousia citriodora) (90-98%), Litsea citrata (90%), Litsea cubeba (70-85%), lemon tea-tree (70-80%), Ocimum gratissimum (66.5%), Lindera citriodora (about 65%), Calypranthes parriculata (about 62%), petitgrain (36%), lemon verbena (30-35%), lemon ironbark (26%), lemon balm (11%), line (6-9%), lemon (2-5%), and orange.

Additionally, citral has been shown to provide thermogenic response by increasing cellular metabolism. Increasing cellular metabolism may provide additional weight-loss results with an otherwise similar diet as the cells burn through calories more quickly.

Other Natural Active Ingredients

Any suitable essential oil can be employed in embodiments of a weight loss and satiation composition provided (1) the essential oil has therapeutic properties (e.g., the essential oil is effective in promoting weight loss), (2) the essential oil remains thermally stable in the composition, and (3) the essential oil is non-toxic to mammals (e.g., humans) and will be suitable for oral administration. Preferably, the thermostability and photostability of the essential oil is over a prolonged period of time, e.g., up to about 3 years, up to about 1 year, or up to about 6 months, typically experienced in the manufacturing, packaging, shipping, and/or storage of the composition. The preferred essential oil will also preferably comply with any controlling or governing body of law.

Suitable specific essential oils may be derived from and include one or more of the following: ajowan, sweet almond, allspice, aloe vera, ammi visnaga (khella), amyris, angelica root, angelica seed, anise, anise seed, star anise, apricot kernel, absolute arnica, avocado, unrefined avocado, Copaiba balsam, balsam Peru genuine, balsam Peru, balsam peru liquid resin, balsam tolu, sweet french basil, basil, basil ct. methyl chavicol, lemon ct. citral basil, sweet ct. linalool basil, bay laurel, bay leaf, bay rum, bay leaf West Indies, bees wax, unrefined bees wax, benzoin absolute, benzoin resinoid, bergamot, mint bergamot, Italian bergamot, free bergaptene bergamot, birch, sweet birch, borage oil, boronia, butter, buchu leaf, cajeput, calamus, calendula oil, infused calendula oil, camellia oil, cannabis, caraway, caraway seed, cardamom, absolute carnation, carrot seed, high carotol carrot seed, carrot seed oil, cassia, cassis bud (black currant), castor oil, catnip, oil of catnip, cedarleaf, western red cedarleaf, cedarwood, Atlas cedarwood, Himalayan cedarwood, Virginia cedarwood, celery seed, chamomile, blue chamomile, German chamomile, Moroccan chamomile, Moroccan wild chamomile, Roman chamomile, champaca, cilantro, true cinnamon bark, cinnamon bark, cinnamon leaf, cinnamon cassia, cistus, citronella, Java citronella, ciste oil, artificial civet, clary sage, high sclareol clary sage, clementine, Italian clementine peel, clove, clove bud, clove leaf, cocoa, cocoa butter, unrefined cocoa butter, coconut, refined coconut, cognac, combava petitgrain, coriander, green coriander, cornmint, costus, cumin, cypress, davana, dill, dill weed, elemi, erigeron (fleabane), eucalyptus citriodora, eucalyptus globulus, lemon eucalyptus, fennel, sweet fennel, fenugreek, fir (i.e. abies ssp.), fir needles (i.e. Canada fir needle, Siberia fir needle, white fir needle, etc.), frankincense, India frankincense, Oman frankincense, galbanum oil, garlic, genet, geranium, geranium leaf, geranium rose, Bourbon geranium, Egyptian geranium, ginger, Cochin extra ginger, ginsing, Siberian ginsing, Korean ginsing, grapefruit, pink grapefruit, white grapefruit, grapeseed, hazelnut, helichrysum, helichrysum immortelle, Mad. helichrysum, Balkan helichrysum, Corsica helichrysum, France helichrysum, hemp oil, absolute honeysuckle, hyssop, hyssop decumbens, absolute immortelle, fragrant aster inula, Jamaican gold, unrefined Jamaican gold, jasmine, absolute jasmine, grandiflorum jasmine, sambac jasmine, jojoba oil, helio-carrot in jojoba, melissa in jojoba, absolute jonquille, juniper (i.e. juniperus ssp), juniper berry, (i.e, Siberia juniper berry, Croatia juniper berry, etc.) lanolin, unrefined anhydrous lanolin, lantana camara, laurel nobilis, lavandin, abrialis lavandin, grosso lavandin, lavender, Oregon lavender, Bulgarian lavender, Russian lavender, high-altitude lavendar, wild-crafted lavender, lavendin, organic lavindin, lemon, lemongrass, lime, distilled lime, expressed lime, litsea, litsea cubeba, blue, pink and white lotus, macadamia oil, mace, green mandarin, red mandarin, yellow mandarin, manuka, absolute marigold, marigold flower, marjoram, Spanish marjoram, sweet marjoram (true), massoia bark, melissa, codistilled melissa, “rectified” melissa, true melissa, absolute mimosa, mimosa, monarda, mugwort, musk seed, myrrh, myrtle, absolute narcissus, neroli (orange blossom), niaouli, nutmeg, extra nutmeg, oakmoss, absolute oak moss, ocotea, olibanum, absolute opopanax, bitter orange, blood orange, sweet orange, wild West Indian orange, oregano, orris root, concrete orris, osmanthus, palm, refined palm, palmarosa, paprika, parsley seed, patchouli, Indian patchouli oil, Indonesian patchouli, peanut, pecan, pennyroyal, pepper, black pepper, super black pepper, peppermint, India peppermint, USA baby mint peppermint, petitgrain (orange leaves), alcoquiana, pine (i.e. pinus ssp), pine needle (i.e., white pine needle, etc.), evening primrose, ravensara anisata, true ravensara, ravensare, ravintsara, redberry, rosalina, rose, rose geranium, rose otto, Bulgarian rose, English rose, Turkish rose, rosehip seed oil, rosemary, rosemary anti-oxidant extract powder, rosemary verbenone, Morocco rosemary, Spain rosemary, rosewood, rosewood oil, rue, sage, white sage, sage dalmatian, sage officinalis, sage triloba, sandalwood, seabuckthorn berry, sesame oil, sesame seed oil, shea butter, unrefined shea butter, spikenard, green spikenard, spruce (i.e., picea ssp.), St. John's wort, styrax resin, tagetes, tangerine, Dancy tangerine, tarragon, tea tree, Australia tea tree, thuja (cedar leaf), thyme, red thyme, thyme ct. linalool, thyme vulgaris, wild thyme, red thyme, mixed tocopherols, tolu balsam resin, absolute tuberose, tuberose, tumeric, valerian, vanilla, pure vanilla extract, vanilla bean, absolute vanilla bourbon, vegetable glycerin, absolute verbena, vetiver, violete leaves, vitex, organic Haiti vetiver, absolute violet leaf, walnut oil, wintergreen, natural wintergreen, wormwood, yarrow, ylang ylang, ylang ylang I, ylang ylang II, ylang ylang III, ylang ylang compound, ylang ylang complete, and ylang ylang extra. Specifically, suitable exemplary essential oils may include citral, cinnamon cassia, ocotea, pomegranate seed oil, or a combination thereof.

Other essential oils and additives may include, laurus nobilis, melaleuca alternifolia, m quienquenervia, m ericifolia, hibiscus abelmoschus, beeswax absolute (apis mellifera), bergamot mint (mentha citrate), bougoin de cassis (ribes nigrum), broom absolute (spartium junceum), cajuput (melaleuca leucadendron), camphor (cinnamomum camphora), cardamon seed (ellettaria cardamomum), cassie absolute (acacia farnesiana), everlasting absolute (helichrysum stoechas), hay absolute, ho wood (cinnamomum camphora), labdanum absolute (cistus ladaniferus), lentisque absolute (pistacia lentiscus), orange flower absolute (citrus aurantium), origanum, savory (satureja montana), spearmint (mentha spicata), tobacco absolute (spp. nicotiana), tonka absolute (dipteryx odorata), treemoss absolute (evernia prunastri), vetyvert (vetivera zizanoides), d limonene, 1 Limonene, mycrene, cineole, menthol, menthone, menthyl acetate, a humulene, gamma humulene, caryophyllene, borneol, linalool, linalyl acetate, methyl salicylate, bornyl acetate, para cymene, eugenal, geraniol, nerol, citronellol, sabinene, alpha pinene, beta pinene, eudesmol, aromadendrebe, globuluol, pterpinen 4 ol, terpinolene, cuminal aldehyde, alpha terpineol, bisaboilol, bisabolol oxides, ocimene, myrcene, fenchol, germacrene D, C, B, a zingiberene, gamma cadinene, beta selinene, farnesol, delta cadinene, alpha selinene, beta selinene, gamma selinene, delta elemene, alpha elemene, piperine, carvone, benzaldehyde, anisyl acetate, anisyl alchohol, camphene, geranyl acetate, isomenthol, isomenthone, vanillin, terpineol, valencene, sinensal, nootketone, gamma terpinene, thymol, carvacrol, a bergamotene, cubebol, merolidol, neryl acetate, methyl eugenol, longifolene, anethole, anisyl acetate, benzyl benzoate, benzyl cinnamate, 5-epiprezizane, khusimene, a-muurolene, khusimone, calacorene, b-humulene, a-longipinene, d-cadinene, valencene, calarene-gurjunene, a-amorphene, epizizanal, 3-epizizanol, khusimol, Isoeugenol, a curcumene, b curcumene, gamma curcumene, perryl alcohol, Iso-khusimol, Valerenol, b-vetivone, a-vetivone, dfN-Butyl-2-Methylbutyrate, Ethyl-2-Methylbutyrate, Isoamyl Isovalerate, Isopropyl-2-Methylbutyrate, 2-Methylbutyric Acid, Methyl-2-Methylbutyrate, Methyl Isovalerate, Phenylethyl Isovalerate, Benzyl Butyrate, Benzyl Isobutyrate, Benzyl Isovalerate, Benzyl Propionate, Butyl Butyrate, Ethyl Isovalerate, Ethyl Valerate, N-Hexyl Isobutyrate, N-Hexyl-2-Methylbutyrate, Isoamyl-2-Methylbutyrate, Isobutyl-2-Methylbutyrate, Phenethyl Butyrate, Phenethyl Isobutyrate, Phenethyl-2-Methylbutyrate, or any combination thereof.

The amount of essential oils (or other compounds) in the penetrating carrier system may be within the effective ranges of the individual oils. For example, in one embodiment a single essential oil may be added to the system within an effective range of 500 ppm to 3500 ppm. In another embodiment, a first essential oil may be added to the system within an effective range of 500 ppm to 3500 ppm, and a second essential oil may be added to the system within an effective range of 100 ppm to 1500 ppm. In another embodiment, a first essential oils may be added to the system within an effective range of 500 ppm to 3500 ppm, a second essential oil may be added to the system within an effective range of 50 ppm to 1500 ppm, and a third essential oil added to the system within an effective range of 50 ppm to 1500 ppm. In another embodiment, a first and second essential oil may be added to the system within an effective range of 500 ppm to 3500 ppm, and a third essential oil added to the system within an effective range of 50 ppm to 1500 ppm. In yet another embodiment, a first, a second, and a third essential oil may be added to the system within an effective range of 500 ppm to 3500 ppm, and a fourth essential oil added to the system within an effective range of 50 ppm to 1500 ppm. Finally, in yet another embodiment, one or more essential oils may be added to the system within an effective range of 500 ppm to 3500 ppm, and one or more additional essential oils added to the system within an effective range of 50 ppm to 1500 ppm.

Exemplary weight loss compositions can be produced by a variety of methods. In some embodiments, an effective amount of each of the components described above may be preformulated in separate solutions. Thereafter, the components may be mixed in effective ratios, as discussed above. Any optionally added ingredients, such as flavorings, dietary supplements, rheology modifiers, emulsifiers, colorants, and the like can be preferably added according to the ratios discussed above.

Weight Loss System

Some embodiments of a weight loss system may include two elements, the combination of the first and second elements being effective in promoting weight loss. The first element may be designed for administration prior to ingesting a meal. The second element may be designed for administration following ingestion of a meal. The various components of the first and second elements may be designed to curb appetite, and maximize weight loss through fat blocking, fat growth suppression, thermogenic increase, and insulin sensitizing.

In some embodiments, a first element may be provided, which includes a fat blocker component, a filler component, and an insulin sensitizer component. The first element may generally be presented in a dry, powder form which is ingested by the user prior to the user consuming a meal. For example, the first element may be encapsulated within a gelcap which may be taken by the user with water. The dry, powder form of the first element may be important to prevent pre-expansion of the filler component prior to administration of the first element to the user. Further, encapsulation of the first element within a gelcap may be desirable, as a gelcap does not require the addition of water or other liquids in preparing the first element.

At least one benefit of the filler component of the first element may be to reduce empty space within the user stomach prior to the user consuming food. Accordingly, the first element may be taken with water or beverage to dissolve the gelcap and expose the filler component to the water. This in turn causes the filler component to expand and reduce the space within the user's stomach. Thus, the filler component may trigger the brain to reduce the appetite of the user, thereby limiting subsequent caloric intake of the user.

At least one benefit of the insulin sensitizer component may be to increase and promote insulin activity within the user prior to caloric intake. The insulin sensitizer component may thereby reduce serum glucose within the user prior to the introduction of additional glucose into the user's blood. Thus, the user's body may be prepared to receive them and metabolize additional glucose as a result of subsequent caloric intake. This reduction in serum glucose may prevent excess glucose buildup within the user's blood which may lead to the conversion of glucose to stored fat.

In some embodiments, a second element may be provided, which includes a fat growth suppressant component, thermogenic increase effect, and a fat blocker component. The second element may include a liquid form which is ingested by the user during or subsequent to the user consuming a meal. For example, the second element may include a beverage which the user consumes with the user's meal. Alternatively, the second element may include a beverage which the user consumes following the user's meal. The second element may further include a powder form which is mixed with water or another liquid prior to ingestion. Further still, the second element may include a powder or liquid form which is provided in a capsule or as a tablet or gelcap.

At least one benefit of the fat growth suppressing component may be to suppress the maturation of adipocyte cells following caloric intake. Further, at least one benefit of the fat blocker component may be to promote fat burning within fat cells in white adipose tissue by increasing the expression of thermogenin following caloric intake. Thus, the combination of the first and second elements work to control caloric intake and assists the user's body in efficiently metabolizing the new calories.

### EXAMPLES

### Example 1

### First Element

Some embodiments may include a weight loss and satiation system which includes a first element comprising fucoxanthin, glucomannan, and cinnamon.

### Example 2

### First Element

Some embodiments may include a weight loss and satiation system which includes a first element comprising fucoxanthin, konjac root, and cinnamon.

### Example 3

### First Element

Some embodiments may include a weight loss and satiation system which includes a first element comprising fucoxanthin, glucomannan, and cassia.

### Example 4

### First Element

Some embodiments may include a weight loss and satiation system which includes a first element comprising fucoxanthin, konjac root, and cassia.

### Example 5

### First Element

Some embodiments may include a weight loss and satiation system which includes a first element comprising fucoxanthin, konjac root, and ocotea.

### Example 6

### Second Element

Some embodiments may include a weight loss and satiation system which includes a second element comprising a citral, and punicic acid.

### Example 7

### Second Element

Some embodiments may include a weight loss and satiation system which includes a second element comprising lemongrass, and punicic acid.

### Example 8

### Weight Loss and Satiation System

Some embodiments may include a weight loss system which includes a first element as disclosed in Example 1, above, used in combination with a second element as disclosed in Example 6, above.

### Example 9

### Weight Loss System

Some embodiments may include a weight loss system which includes a first element as disclosed in Example 1, above, used in combination with a second element as disclosed in Example 6, above.

### Example 10

### Weight Loss System

Some embodiments may include a weight loss system which includes a first element as disclosed in Example 2, above, used in combination with a second element as disclosed in Example 6, above.

### Example 11

### Weight Loss System

Some embodiments may include a weight loss system which includes a first element as disclosed in Example 2, above, used in combination with a second element as disclosed in Example 7, above.

### Example 12

### Weight Loss System

Some embodiments may include a weight loss system which includes a first element as disclosed in Example 3, above, used in combination with a second element as disclosed in Example 6, above.

### Example 13

### Weight Loss System

Some embodiments may include a weight loss system which includes a first element as disclosed in Example 3, above, used in combination with a second element as disclosed in Example 7, above.

### Example 14

### Weight Loss System

Some embodiments may include a weight loss system which includes a first element as disclosed in Example 4, above, used in combination with a second element as disclosed in Example 6, above.

### Example 15

### Weight Loss System

Some embodiments may include a weight loss system which includes a first element as disclosed in Example 4, above, used in combination with a second element as disclosed in Example 7, above.

### Example 16

### Weight Loss System

Some embodiments may include a weight loss system which includes a first element as disclosed in Examples 1 and 2, above, used in combination with a second element as described in Example 6, above.

### Example 17

### Weight Loss System

Some embodiments may include a weight loss system which includes a first element as disclosed in Examples 1 and 2, above, used in combination with a second element as described in Example 7, above.

### Example 18

### Weight Loss System

Some embodiments may include a weight loss system which includes a first element as disclosed in Example 5, above, used in combination with a second element as disclosed in Example 6, above.

### Example 19

### Weight Loss System

Some embodiments may include a weight loss system which includes a first element as disclosed in Example 5, above, used in combination with a second element as disclosed in Example 7, above.

### Example 20

### Weight Loss System

Some embodiments may include a weight loss system which includes a first element comprising one or more of the elements disclosed in Examples 1, 2, 3, 4, and 5 above, used in combination with a second element comprising one or more of the elements disclosed in Examples 6 and 7, above.

Other specific forms and combination of weight loss systems may be performed and provided without departing from its structures, methods, or other essential characteristics as broadly described herein and claimed hereinafter. The described embodiments are to be considered in all respects only as illustrative, and not restrictive. The scope is, therefore, indicated by the appended claims, rather than by the foregoing description. All changes that come within the meaning and range of equivalency of the claims are to be embraced within their scope.

What is claimed is:

1. A composition for promoting weight loss, comprising:
a fat blocker;
a filler component;
an insulin sensitizer; and
a fat growth suppressant.

2. The composition of claim 1, wherein the fat blocker is a carotenoid.

3. The composition of claim 1, wherein the fat blocker is selected from the group consisting of flucoxanthin and punicic acid.

4. The composition of claim 1, wherein the filler component is a high-fiber composition.

5. The composition of claim 1, wherein the filler component is selected from the group consisting of konjac root and glucomannan.

6. The composition of claim 1, wherein the insulin sensitizer is selected from the group consisting of cinnamon, Cinnamonum cassia, cassia, and punicic acid.

7. The composition of claim 1, wherein the insulin sensitizer is selected from a group consisting of ocotea, cloves, bay leaves, turmeric, bitter melon, Gymnema, Korean ginsing, onions, garlic, flaxseed meal, α-lipoic acid, biotin, carnitine, vanadium, chromium, magnesium, zinc, vitamin B₃, vitamin E, and vitamin K.

8. The composition of claim 1, wherein the fat growth suppressant is selected from the group consisting of citral and lemongrass.

9. A weight loss system, comprising:
a first element comprising:
a fat blocker;
a filler component; and
a first insulin sensitizer; and
a second element comprising:
a fat growth suppressant; and
a second insulin sensitizer.

10. The weight loss system of claim 9, wherein the fat blocker is a carotenoid.

11. The weight loss system of claim 9, wherein the first element comprises a dry powder form.

12. The weight loss system of claim 9, wherein the second element comprises a liquid form.

13. The weight loss system of claim 9, wherein the fat blocker is selected from the group consisting of flucoxanthin and punicic acid.

14. The weight loss system of claim 9, wherein the filler component is selected from the group consisting of konjac root and glucomannan.

15. The weight loss system of claim 9, wherein the fat growth suppressant is selected from the group consisting of citral and lemongrass.

16. The weight loss system of claim 9, wherein the first element is taken prior to consuming a meal.

17. The weight loss system of claim 9, wherein the second element is taken following consumption of a meal.

18. The weight loss system of claim 9, wherein the insulin sensitizer is selected from the group consisting of cloves, bay leaves, turmeric, bitter melon, Gymnema, Korean ginsing, onions, garlic, flaxseed meal, α-lipoic acid, biotin, carnitine, vanadium, chromium, magnesium, zinc, vitamin B₃, vitamin E, and vitamin K.

19. The weight loss system of claim 9, wherein the filler component is a high-fiber composition.

20. A weight loss system, comprising a kit including a first and second element according to claim 9.

21. The weight loss system of claim 9, wherein the second element includes a thermogenic increase component.
